(19) United States
(12) Patent Application Publication
(10) Pub. No.: US 2006/0030556 A1
(43) Pub. Date: Feb. 9, 2006

(54) Neurokinin-1 receptor antagonists for the treatment of conditions responsive to testosterone elevation, including testosterone deficiency

(75) Inventors: Eberhard K.M. Fuchs, Weesp (NL); Boldizsar Czeh, Weesp (NL); Mayke B. Hesselink, Weesp (NL); Hubert Thole, Weesp (NL); Martinus Th M. Tulp, Weesp (NL)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: SOLVAY PHARMACEUTICALS B.V.

(21) Appl. No.: 11/193,361

(22) Filed: Aug. 1, 2005

Related U.S. Application Data
(60) Provisional application No. 60/598,422, filed on Aug. 4, 2004.

(51) Int. Cl. A61K 31/551; A61K 31/498; A61K 31/5377

(52) U.S. Cl. 514/218; 514/249; 514/254.05; 514/254.09; 514/234.5

(57) ABSTRACT

The invention relates to the use of neurokinin-1 receptor antagonists as testosterone replacement therapy. In a further aspect the invention relates to the use of neurokinin-1 receptor antagonists for the preparation of medicaments for treating conditions associated with low testosterone levels, in patients having deficient testosterone levels. In another aspect the invention relates to the use of neurokinin-1 receptor antagonists for the preparation of medicaments for treating hypogonadism in men.

The invention relates to the use of neurokinin-1 receptor antagonists as testosterone replacement therapy. In a further aspect the invention relates to the use of neurokinin-1 receptor antagonists for the preparation of medicaments for treating conditions associated with low testosterone levels, in patients having deficient testosterone levels. In another aspect the invention relates to the use of neurokinin-1 receptor antagonists for the preparation of medicaments for treating hypogonadism in men.

Testosterone

Testosterone, the principal androgen, is synthesized in the testis, the ovary, and the adrenal gland. When diminished or absent from the body, pathological conditions can arise in the body due to a testosterone deficiency.

Testosterone in Men

Testosterone is the major circulating androgen in men. More than 95% of the hormone produced is secreted by the Leydig cells in the testes. For the development and maintenance of testicular function two hormones are required: luteinizing hormone (LH) and follicle stimulating hormone (FSH), both produced by the pituitary gland. The most important hormone for the regulation of Leydig cell number and function is LH. In eugonadal men, LH secretion from the pituitary is inhibited through a negative-feedback pathway by increased concentrations of testosterone through the inhibition of the release of gonadotropin-releasing hormone (GRH) by the hypothalamus. FSH promotes spermatogenesis and is essential for the normal maturation of sperm. FSH secretion from the pituitary normally is inhibited through a negative-feedback pathway by increased testosterone concentrations. Testosterone is responsible primarily for the development and maintenance of secondary sex characteristics in men. Testosterone circulates in the blood 98% bound to protein. In men, approximately 40% of the binding is to the high-affinity sex hormone binding globulin (SHBG). The remaining 60% is bound weakly to albumin. There is considerable variation in the half-life of testosterone reported in the literature, ranging from 10 to 100 minutes. Researchers do agree, however, that circulating testosterone has a diurnal variation in normal young men. Maximum levels occur at approximately 6:00 to 8:00 a.m. with levels declining throughout the day.

Hypogonadal Men and Current Treatments for Hypogonadism

Male hypogonadism results from a variety of patho-physiological conditions in which testosterone concentration is diminished below the normal range. Hypogonadism is generally classified into one of three types.

Primary hypogonadism includes the testicular failure due to congenital or acquired anorchia, XYY Syndrome, XX males, Noonan's Syndrome, gonadal dysgenesis, Leydig cell tumors, maldescended testes, varicocele, Sertoli-Cell-Only Syndrome, cryptorchidism, bilateral torsion, vanishing testis syndrome, orchiectomy, Klinefelter's Syndrome, chemotherapy, toxic damage from alcohol or heavy metals, and general disease (renal failure, liver cirrhosis, diabetes, myotonia dystrophica). Patients with primary hypogonadism show an intact feedback mechanism in that the low serum testosterone concentrations are associated with high FSH and LH concentrations. However, because of testicular or other failures, the high LH concentrations are not effective at stimulating testosterone production.

Secondary hypogonadism involves an idiopathic gonadotropin or LH-releasing hormone deficiency. This type of hypogonadism includes Kallman's Syndrome, Prader-Labhart-Willi's Syndrome, Laurence-Moon-Biedl's Syndrome, pituitary insufficiency/adenomas, Pasqualini's Syndrome, hemochromatosis, hyperprolactin-emia, or pituitary-hypothalamic injury from tumors, trauma, radiation, or obesity. Because patients with secondary hypogonadism do not demonstrate an intact feedback pathway, the lower testosterone concentrations are not associated with increased LH or FSH levels. Thus, these men have low testosterone serum levels but have gonadotropins in the normal to low range.

Geriatric hypogonadism is age-related. Men experience a slow but continuous decline in average serum testosterone after approximately age 20 to 30 years. It is estimated that the decline is about 1-2% per year. Cross-sectional studies in men have found that the mean testosterone value at age 80 years is approximately 75%, of that at age 30 years. Because the serum concentration of SHBG increases as men age, the fall in bioavailable and free testosterone is even greater than the fall in total testosterone. Researchers have estimated that approximately 50% of healthy men between the ages of 50 and 70 have levels of bioavailable testosterone that are below the lower normal limit. Moreover, as men age, the circadian rhythm of testosterone concentration is often muted, dampened, or completely lost. The major problem with aging appears to be within the hypothalamic-pituitary unit. For example, it has been found that with aging, LH levels do not increase despite the low testosterone levels. Regardless of the cause, these untreated testosterone deficiencies in older men may lead to a variety of physiological changes commonly referred to as “male menopause”.

Hypogonadism is the most common hormone deficiency, affecting 5 in every 1,000 men. It is estimated that less than five percent of the men of all ages with hypogonadism currently receive testosterone replacement therapy. Thus, for years, methods of delivering testosterone to men have been investigated. These methods include intramuscular injections, oral replacement, pellet implants, and transdermal patches. All of these methods suffer from one or more drawbacks such as polycythemia (and increased risk of stroke), gynecomastia, prostatic enlargement, hormone dependent cancer at high doses, sodium and water retention, impairment of hepatic function, hypercholeresteremia, suppression of high density lipoprotein concentrations and modulation of lipoprotein composition. Specifically, subdermal implants, used as a method of testosterone replacement since the 1940s, require a surgical procedure which many hypogonadal men simply do not wish to endure. Implant therapy includes a risk of extrusion, bleeding, infection or scarring. Perhaps most important, the pharmacokinetic profile of testosterone pellet implant therapy fails to provide men with a suitable consistent testosterone level. Patients receiving testosterone ester injection treatments, applied since the 1950s, often complain that the delivery mechanism is painful and causes local skin reactions. It is also inconvenient because injection usually requires the patient to visit his physician every two to three weeks. Finally, injection-based testosterone replacement treatments still create an undesirable pharmacokinetic profile. Oral, sublingual, or buccal preparations of androgens have been used as a means for testosterone replacement since the 1970s. But their pharmacokinetic profiles are also undesirable because patients are subjected to super-physiologic testosterone levels followed by a quick return to the baseline. Transdermal testosterone patches generally offer an improved pharmacokinetic profile compared to other currently used testosterone delivery mechanisms. However the clinical and survey data shows that all of these patches suffer from significant drawbacks, such as itching, burn-like blisters, and erythema.

Moreover, one recent study has concluded that the adverse effects associated with transdermal patch systems are “substantially higher” than reported in clinical trials (see Parker et al., “Experience with transdermal testosterone replacement therapy for hypogonadal Men, Clin. Endocrinol. (Oxf), 50(1), 57-62 (1999)). Thus, the transdermal patch still remains an inadequate testosterone replacement therapy alternative for most men.

Testosterone in Women

Decreased production of testosterone by women can be caused by several factors, including the use of oral contraceptives, surgery, e.g. removal of the uterus (hysterectomy), removal of one or both ovaries (ovariectomy); estrogen replacement therapy in post-menopausal women, premature ovarian failure, adrenal dysfunction, for instance primary adrenal insufficiency, corticosteroid-induced adrenal suppression, panhypopituitarrism, and chronic illness such as systemic lupus erythematosis, rheumatoid arthritis, human immunodeficiency virus infection, chronic obstructive lung disease and end stage renal disease.

In women, testosterone deficient disorders: disorders, conditions or diseases due to lack of endogenous testosterone production or utilization thereof, include hypogonadism, hypercholesterolemia, abnormal electrocardiograms, vasomotor symptoms, diabetic retinopathy, hyperglycemia, hyperinsulinemia, hypoinsulinemia, increase percentage of body fat, hypertension, obesity, osteoporosis, osteopenia, dysphoria, decreased muscle strength, fatigue, vaginal dryness, thinning of the vaginal wall, menopausal symptoms and hot flashes, cognitive dysfunction, cardiovascular disease, central nervous system disorders, Alzheimer's disease, dementia, cataracts, and cervical cancer, uterine cancer or breast cancer.

The goal of the present invention is to develop a therapy for testosterone deficiency alternative to the supply of exogenous testosterone, i.e. a therapy aimed at elevation of endogenous testosterone levels.

Surprisingly, It has been found that neurokinin-1 receptor antagonists induce a profound increase in serum testosterone levels in animals subjected to chronic psychosocial stress. This property makes them useful as testosterone replacement therapy, and for the preparation of medicaments for treating conditions associated with low testosterone levels, in patients having deficient testosterone levels, as well as for the preparation of medicaments for treating hypogonadism in men.

The invention further relates to the use of compounds of the general formula (I) as testosterone replacement therapy: 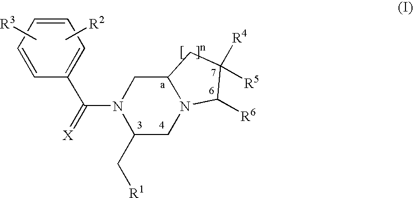
wherein:
R¹ represents phenyl, 2-indolyl, 3-indolyl, 3-indazolyl or benzo[b]thiophen-3-yl, which groups may be substituted with halogen or alkyl (1-3C),
R² and R³ independently represent halogen, H, OCH₃, CH₃ and CF₃,
R⁴, R⁵ and R⁶ independently represent H, OH, O-alkyl(1-4C), CH₂OH, NH₂, dialkyl(1-3C)N, pyrrolidin-1-yl, piperidin-1-yl, morpholin-4-yl or morpholin-4-yl substituted with one or two methyl or methoxymethyl groups, morpholin-4-ylamino, morpholin-4-ylmethyl, imidazol-1-yl, thiomorpholin-4-yl, 1,1-dioxo-thiomorpholin-4-yl or 3-oxa-8-azabicyclo[3.2.1]oct-8-yl; R⁴ and R⁵ together may represent a keto, a 1,3-dioxan-2-yl or a 1,3-dioxolan-2-yl group,
X represents either O or S,
n has the value of 1, 2 or 3,
a is the asymmetrical carbon atom 8a, 9a or 10a when n equals 1, 2 or 3 respectively,

The invention further relates to the use of compounds of the general formula (II) as testosterone replacement therapy: 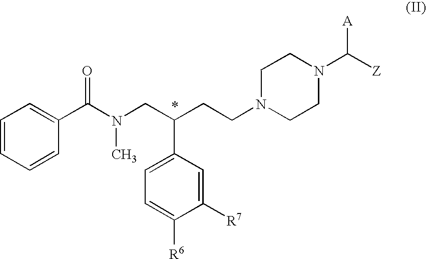
wherein
A is naphthyl, phenyl optionally substituted by hydroxy, mono- or bicyclic heteroaryl or C₃₋₆-alkenyl optionally substituted by phenyl,
Z stands for a subgroup of the general formula 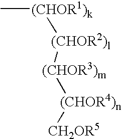
   wherein
R¹ is hydrogen or lower alkanoyl, or together with another substituent, selected from the group consisting of R², R³, R⁴ and R⁵, may form a 5- or 6-ring bridged by carbonyl, thiocarbonyl or by methylene optionally substituted by lower alkyl or C₄₋₅-alkylene,
R² is hydrogen or lower alkanoyl, or together with another substituent, selected from the group consisting of R¹, R³, R⁴ and R⁵, may form a 5- or 6-ring bridged by carbonyl, thiocarbonyl or by methylene optionally substituted by lower alkyl or C₄₋₅-alkylene,
R³ is hydrogen or lower alkanoyl, or together with another substituent, selected from the group consisting of R¹, R², R⁴ and R⁵, may form a 5- or 6-ring bridged by carbonyl, thiocarbonyl or by methylene optionally substituted by lower alkyl or C₄₋₅-alkylene,
R⁴ is hydrogen or lower alkanoyl, or together with another substituent, selected from the group consisting of R¹, R², R³ and R⁵, may form a 5- or 6-ring bridged by carbonyl, thiocarbonyl or by methylene optionally substituted by lower alkyl or C₄₋₅-alkylene,
R⁵ is hydrogen or lower alkanoyl, or together with another substituent, selected from the group consisting of R¹, R², R³ and R⁴, may form a 5- or 6-ring bridged by carbonyl, thiocarbonyl or by methylene optionally substituted by lower alkyl or C₄₋₅-alkylene,
k is 0 or 1,
l is 0 or 1,
m is 0 or 1,
n is 0 or 1,
R⁶ is halogen or hydrogen, and
R⁷ is halogen or hydrogen,

The invention further relates to the use of compounds of the general formula (III) as testosterone replacement therapy: 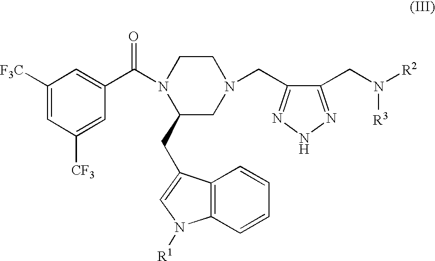
wherein
R¹ is hydrogen or lower alkyl,
R² is lower alkyl, di-lower-alkylamino lower alkyl, lower-alkoxycarbonyl lower alkyl; cyclo(hetero)alkyl having 5-6 ring atoms, which may optionally be substituted once or twice by lower alkyl and which optionally contains 1-2 double bonds; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl or by spiro-C₄-C₅-alkylene; or phenyl lower alkoxy optionally substituted once or twice in the phenyl ring by halogen, lower alkyl and/or lower alkoxy, and
R³ is lower alkyl, lower-alkoxycarbonyl lower alkyl or cyclo(hetero)alkyl with 5-6 ring atoms which is optionally substituted once or twice by lower alkyl, or
R² and R³, together with the nitrogen to which they are bonded, form a cyclic group of formula a, 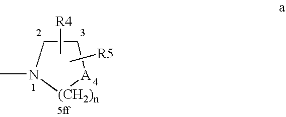
   wherein
A is nitrogen, oxygen, methylene or methylidene, the double bond of which, together with the adjacent carbon, is formed in position 3 of group a,
n is a whole number from 1 to 3,
R⁴ is hydrogen, lower alkyl, lower-alkoxy lower alkyl, lower alkoxycarbonyl, lower-alkoxycarbonyl lower alkyl, di-lower-alkylamino lower alkyl; (hetero)phenyl optionally substituted once or twice by halogen, lower alkyl and/or lower alkoxy; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl; cyclo(hetero)alkyl with 5-6 ring atoms, or cyclo(hetero)alkyl lower alkyl, the cyclo(hetero)alkyl group of which has 5-6 ring atoms, and
R⁵ is hydrogen, lower alkyl or lower-alkoxy lower alkyl, or
R⁴ and R⁵ together are spiroethylenedioxy bonded to a carbon of group a; C₃-C₄-alkylene bonded to two adjacent atoms of group a; or phenyl fused via two adjacent carbons of group a, or
R² and R³, together with the nitrogen to which they are bonded, form a pyrrolidine ring which is substituted twice by C₄-alkylene which is bonded each time via two adjacent carbon atoms,

The invention further relates to the use of compounds of the general formula (IV) as testosterone replacement therapy: 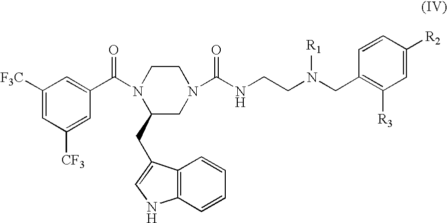
wherein
R₁ is hydrogen or lower alkyl,
R₂ is hydrogen or halogen and
R₃ is hydrogen or lower alkoxy,

The invention further relates to the use of compounds of the general formula (V) as testosterone replacement therapy: 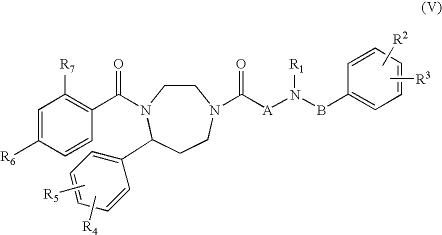
wherein
R¹ is hydrogen or lower alkyl,
R² is hydrogen, lower alkyl, lower alkoxy, halogen or trifluoromethyl, and
R³ is hydrogen, lower alkyl, lower alkoxy, halogen or trifluoromethyl, or
R² and R³ together are alkylenedioxy with 1 to 2 carbon atoms, bonded to adjacent carbon atoms of the phenyl ring,
R⁴ is hydrogen, lower alkyl, lower alkoxy, halogen or trifluoromethyl, and
R⁵ is hydrogen, lower alkyl, lower alkoxy, halogen or trifluoromethyl, or
R⁴ and R⁵ together are alkylenedioxy with 1 to 2 carbon atoms, bonded to adjacent carbon atoms of the phenyl ring,
R⁶ is lower alkyl, halogen or trifluoromethyl,
R⁷ is lower alkyl, halogen or trifluoromethyl,
A is a —(CH₂)ₙ— group in which n stands for an integer from 1 to 3, or an —NH—(CH₂)ₘ— group in which m stands for an integer from 2 to 3, and
B is an alkylene chain with 1 to 3 carbon atoms, optionally substituted by lower alkyl,

The invention further relates to the use of compounds of the general formula (VI) as testosterone replacement therapy: 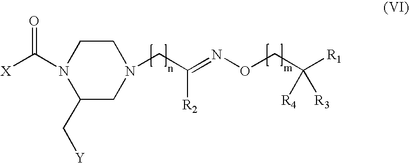
wherein:
X represents phenyl or pyridyl substituted with 1 or 2 substituents from the group CH₃, CF₃, OCH₃, halogen, cyano and 5-CF₃-tetrazol-1-yl
Y represents 2- or 3-indolyl, phenyl, 7-aza-indol-3-yl or 3-indazolyl, 2-naphthyl, 3-benzo[b]thiophenyl or 2-benzofuranyl, which groups may be substituted with one or more halogen or alkyl (1-3C)
n has the value 0-3
m has the value 0-2
R₁ represents NH₂, NH-alkyl (1-3C), dialkyl (1-3C)N, morpholino or morpholino substituted with one or two CH₃ and/or methoxymethyl groups, thiomorpholino 1,1-dioxothiomorpholino, 2-, 3- or 4-pyridyl or 4-CH₃-piperazinyl
R₂ is hydrogen, alkyl (1-4C) or phenyl, or R₂ together with (CH₂)ₘ wherein m is 1, and the intermediate carbon, nitrogen and oxygen atoms forms an isoxazolyl or a 4,5-dihydroisoxazolyl group,
R₃ and R₄ independently represent hydrogen or methyl, or R₃ and R₄ together are oxygen,

The invention further relates to the use of compounds of the general formula (VII) as testosterone replacement therapy: 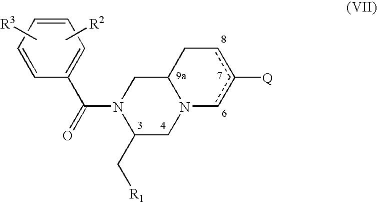
wherein:
R¹ represents phenyl, 2-indolyl, 3-indolyl, 3-indazolyl or benzo[b]thiophen-3-yl, which groups may be substituted with halogen or alkyl (1-3C),
R² and R³ independently represent halogen, H, OCH₃, CH₃ and CF₃,
Q represents an optionally substituted aromatic or heteroaromatic five- or six-membered ring,
the pyrido[1,2-a]pyrazine moiety may or may not contain a double bond between either carbon atoms 6 and 7 or between carbon atoms 7 and 8,

Within the context of this application, the term “lower alkyl” means methyl, ethyl, 1-propyl, isopropyl, 2-methyl-1-propyl, isobutyl, 1-butyl or 2-butyl

The detailed syntheses of the compounds of the invention with the general structures (I)-(VII) are known from WO 03/050086 (I) U.S. 20030125557 (II), EP 1 176 144 (III), U.S. Pat. No. 6,001,833 (IV) U.S. Pat. No. 6,040,303 (V), WO 03/006459 (VI), EP 03 103566.0 (VII)

To the invention also belongs the use of neurokinin-1 receptor antagonists described in the following patents and patent applications as testosterone replacement therapy:
EP-0360390, EP-0394989, EP-0428434, EP-0429366, EP-0430771, EP-0436334, EP-0443132, EP-0474561, EP-0482539, EP-0498069, EP-0512901, EP-0512902, EP-0514273, EP-0514274, EP-0514275, EP-0514276, EP-0517589, EP-0520555, EP-0522808, EP-0528495, EP-0532456, EP-0533280, EP-0536817, EP-0545478, EP-0559538, EP-0566069, EP-0577394, EP-0590152, EP-0599538, EP-0610793, EP-0615751, EP-0627221, EP-0634402, EP-0646372, EP-0652218, EP-0655246, EP-0659409, EP-0680749, EP-0684257, EP-0686629, EP-0693489, EP-0694535, EP-0699655, EP-0699674, EP-0707006, EP-0708101, EP-0709375, EP-0709376, EP-0714891, EP-0715855, EP-0717997, EP-0717998, EP-0721778, EP-0722722, EP-0722736, EP-0723959, EP-0733632, EP-0737471, EP-0737478, EP-0739892, EP-0747055, EP-0769300, EP-0770391, EP-0773026, EP-0776893, EP-0780375, EP-0790254, EP-0791592, EP-0824100, EP-0829480, EP-0867182, EP-0873753, EP-0898967, EP-0899270, EP-0962457, EP-0992493, EP-1027892, EP-1035115, EP-1095655, EP-1095939, EP-1099446, EP-1103545, EP-1103546, EP-1172106, EP-1186318, EP-1192952, EP-1295599,
GB-2266529, GB-2268931, GB-2269170, GB-2269590, GB-2271774, GB-2282807, GB-2287404, GB-2292144, GB-2293168, GB-2293169, GB-2302689, GB-2304714, GB-2311523, GB-2318293, GB-2321058, GB-2347423,
JP-07126202, JP-08081470, JP-08337583, JP-09040662, JP-09104674, JP-09124609, JP-10109989, JP-10182650, JP-10273489, JP-10298197, JP-11043435, JP-11043489, JP-11140052, JP-11246559, JP-11286443, JP-1±1349482, JP-2000026424, JP-2000103793, JP-2003277263, JP-2003277384, JP-2003321461,
U.S. Pat. No. 4,839,465, U.S. Pat. No. 5,216,163, U.S. Pat. No. 5,256,671, U.S. Pat. No. 5,338,845, U.S. Pat. No. 5,393,762, U.S. Pat. No. 5,422,354, U.S. Pat. No. 5,491,140, U.S. Pat. No. 5,498,614, U.S. Pat. No. 5,554,633, U.S. Pat. No. 5,563,133, U.S. Pat. No. 5,604,252, U.S. Pat. No. 5,607,947, U.S. Pat. No. 5,683,725, U.S. Pat. No. 5,728,686, U.S. Pat. No. 5,750,549, U.S. Pat. No. 5,789,422, U.S. Pat. No. 5,891,875, U.S. Pat. No. 6,051,707, U.S. Pat. No. 6,051,717, U.S. Pat. No. 6,063,926, U.S. Pat. No. 6,156,749, U.S. Pat. No. 6,262,077, U.S. Pat. No. 6,479,486, U.S. Pat. No. 6,555,552,
WO-09005525, WO-09005729, WO-09107179, WO-09109844, WO-09118878, WO-09118899, WO-09201688, WO-09206079, WO-09212151, WO-09212152, WO-09215585, WO-09217449, WO-09220661, WO-09220676, WO-09221677, WO-09300330, WO-09300331, WO-09301159, WO-09301160, WO-09301165, WO-09301169, WO-09301170, WO-09306099, WO-09309116, WO-09310073, WO-09311110, WO-09314113, WO-09318002, WO-09318023, WO-09319064, WO-09321154, WO-09321155, WO-09321181, WO-09321215, WO-09323380, WO-09324465, WO-09400440, WO-09401402, WO-09402461, WO-09403429, WO-09403445, WO-09404494, WO-09404496, WO-09405625, WO-09407843, WO-09410167, WO-09410168, WO-09410170, WO-09411368, WO-09413639, WO-09413646, WO-09413663, WO-09413694, WO-09414767, WO-09415903, WO-09416697, WO-09419320, WO-09419323, WO-09420126, WO-09420500, WO-09422822, WO-09426735, WO-09426740, WO-09427966, WO-09439309, WO-09500536, WO-09502595, WO-09504040, WO-09504042, WO-09504551, WO-09506645, WO-09507886, WO-09507908, WO-09508549, WO-09511880, WO-09511895, WO-09514017, WO-09515311, WO-09516679, WO-09517382, WO-09518124, WO-09518129, WO-09519344, WO-09519966, WO-09520575, WO-09521819, WO-09522525, WO-09523798, WO-09526338, WO-09528389, WO-09528418, WO-09529912, WO-09530674, WO-09530687, WO-09533744, WO-09535279, WO-09605181, WO-09605193, WO-09605203, WO-09606094, WO-09607649, WO-09608480, WO-09610562, WO-09610568, WO-09612479, WO-09614845, WO-09618643, WO-09616939, WO-09620197, WO-09621661, WO-09624582, WO-09626183, WO-09628441, WO-09629304, WO-09629317, WO-09629326, WO-09629328, WO-09630367, WO-09631214, WO-09632385, WO-09634857, WO-09634864, WO-09637207, WO-09637489, WO-09639383, WO-09639386, WO-09640079, WO-09701553, WO-09701554, WO-09702824, WO-09703066, WO-09705110, WO-09708144, WO-09708166, WO-09713514, WO-09714671, WO-09716440, WO-09717362, WO-09718206, WO-09719074, WO-09719084, WO-09719942, WO-09721702, WO-09724324, WO-09724350, WO-09724356, WO-09730055, WO-09730056, WO-09730989, WO-09730990, WO-09731635, WO-09733873, WO-09738692, WO-09745119, WO-09749710, WO-09801450, WO-09802158, WO-09807694, WO-09807722, WO-09814193, WO-09815277, WO-09817276, WO-09817660, WO-09818465, WO-09818466, WO-09818761, WO-09818785, WO-09818788, WO-09820010, WO-09824438, WO-09824439, WO-09824440, WO-09824441, WO-09824442, WO-09824443, WO-09824444, WO-09824445, WO-09824446, WO-09824447, WO-09827085, WO-09827086, WO-09828297, WO-09831704, WO-09835662, WO-09835663, WO-09843639, WO-09845262, WO-09847513, WO-09847514, WO-09849170, WO-09850368, WO-09854187, WO-09854191, WO-09857954, WO-09900368, WO-09901451, WO-09907373, WO-09907374, WO-09907375, WO-09907376, WO-09907413, WO-09909987, WO-09921823, WO-09924423, WO-09925364, WO-09925714, WO-09926924, WO-09927938, WO-09936424, WO-09947132, WO-09952903, WO-09956550, WO-09959583, WO-09959635, WO-09959972, WO-09961025, WO-09962893, WO-09962900, WO-09964006, WO-09964007, WO-09964008, WO-09964009, WO-09964010, WO-09964011, WO-09965900, WO-00006544, WO-00006571, WO-00006572, WO-00006578, WO-00006580, WO-00007598, WO-00010545, WO-00014109, WO-00020003, WO-00020389, WO-00032192, WO-00034243, WO-00037462, WO-00039114, WO-00047572, WO-00050401, WO-00051984, WO-00053572, WO-00056727, WO-00059873, WO-00068224, WO-00069438, WO-00071107, WO-00071538, WO-00073278, WO-00073279, WO-00073303, WO-00073304, WO-00078759, WO-00124791, WO-00124792, WO-00125219, WO-00125233, WO-00130348, WO-00131020, WO-00132656, WO-00144200, WO-00146167, WO-00146176, WO-00152844, WO-00152854, WO-00164223, WO-00174392, WO-00177069, WO-00177089, WO-00177100, WO-00187838, WO-00187866, WO-00190083, WO-00194346, WO-00195904, WO-00196315, WO-00196320, WO-00196332, WO-00200631, WO-00208232, WO-00210194, WO-00212168, WO-00216324, WO-00216344, WO-00222574, WO-00224629, WO-00224673, WO-00226260, WO-00226724, WO-00232865, WO-00232866, WO-00234699, WO-00242280, WO-00247663, WO-02051440, WO-02051807, WO-02055518, WO-02057250, WO-02062784, WO-02069944, WO-02074771, WO-02079134, WO-02085458, WO-02092604, WO-02102372, WO-03006016, WO-03008396, WO-03009898, WO-03011860, WO-03015784, WO-03018587, WO-03022254, WO-03022839, WO-03030907, WO-03037334, WO-03042173, WO-03050123, WO-03051840, WO-03051868, WO-03053957, WO-03062245, WO-03066589, WO-03066621, WO-03066635, WO-03074462, WO-03084955, WO-03089429, WO-03091226, WO-03091227, WO-03101459, WO-04000355, WO-04004722, WO-04004724, WO-04005255, WO-04005256, WO-04009573, WO-04014850, WO-04020411, WO-04022002, WO-04022539, WO-04024714, WO-04030629, WO-04030695, WO-04031185, WO-04031190, WO-04031191, WO-04032875, WO-04035587, WO 04043908 and WO 04047833.

Particularly, the invention relates to the use of the following NK1 antagonists as testosterone replacement therapy: anthrotainin, aprepitant, AVE-5883, BIIF-1149, BL-1832, BL-1833, CAM-2445, CAM-6108, capsazepine, CGP-47899, CGP-49823, CGP-73400, cizolirtine, CJ-17493, CP-0364, CP-0578, CP-100263, CP-122721, CP-96345, CP-98984, CP-99994, dapitant, DNK-333, E-6006, ezlopitant, FK-224, FK-355, FK-888, FR-113680, GR-138676, GR-203040, GR-71251, GR-82334, GW-597599, GW-679769, GW-823296, isbufylline, KRP-103, L-161644, L-161664, L-709210, L-732138, L-733060, L-736281, L-737488, L-740141, L-741671, L-742311, L-742694, L-743986, L-756867, L-758298, lanepitant, LY-297911, LY-306740, MDL-105172A, MEN-10930, MEN-11149, MEN-11467, NIP-530, NKP-608, nolpitantium besilate, PSI-697, R-1124, R-116301, R-673, RP-67580, RP-73467, RPR-107880, RPR-111905, S-16474, S-18523, S-19752, Sch-60059, SDZ-NKT-343, SP-PE toxin, SSR-240600, TAK-637, TKA-457, vofopitant, WIN-51708, WIN-64745, WIN-64821, WIN-66306, WIN-67689, WIN-68577, WS-9326A, YM-44778, YM-49244, ZD-4794 and ZD-6021

More in particular the invention relates to the use of compounds of formulae: 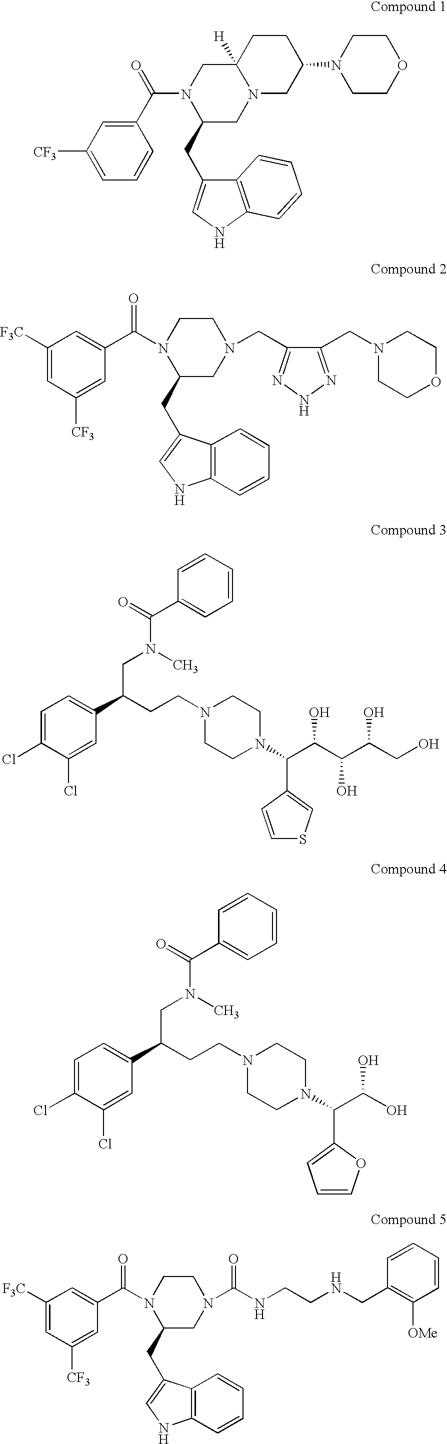 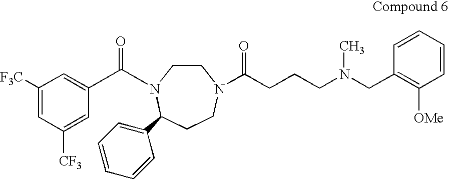
as testosterone replacement therapy.

Most preferred is the use of compound 1 as testosterone replacement therapy.

Pharmaceutical Preparations

The compounds of the invention can be brought into forms suitable for administration by means of usual processes using auxiliary substances such as liquid or solid carrier material. The pharmaceutical compositions of the invention may be administered enterally, orally, parenterally (intramuscularly or intravenously), rectally or locally (topically). They can be administered in the form of solutions, powders, tablets, capsules (including microcapsules), ointments (creams or gel) or suppositories. Suitable excipients for such formulations are the pharmaceutically customary liquid or solid fillers and extenders, solvents, emulsifiers, lubricants, flavorings, colorings and/or buffer substances. Frequently used auxillary substances which may be mentioned are magnesium carbonate, titanium dioxide, lactose, mannitol and other sugars or sugar alcohols, talc, lactoprotein, gelatin, starch, cellulose and its derivatives, animal and vegetable oils such as fish liver oil, sunflower, groundnut or sesame oil, polyethylene glycol and solvents such as, for example, sterile water and mono- or polyhydric alcohols such as glycerol.

Types of pharmaceutical compositions that may be used include but are not limited to tablets, chewable tablets, capsules, solutions, parenteral solutions, suppositories, suspensions, and other types disclosed herein or apparent to a person skilled in the art from the specification and general knowledge in the art. The invention also includes the preparation or manufacture of said pharmaceutical compositions.

In embodiments of the invention, a pharmaceutical pack or kit is provided comprising one or more containers filled with one or more of the ingredients of a pharmaceutical composition of the invention. Associated with such container(s) can be various written materials such as instructions for use, or a notice in the form prescribed by a governmental agency regulating the manufacture, use or sale of pharmaceuticals products, which notice reflects approval by the agency of manufacture, use, or sale for human or veterinary administration.

Animals, Experimental Procedures and Drug Treatment

The experiments were carried out using tree shrews (Tupaia belangen). Experimentally naive adult male tree shrews were obtained from the breeding colony at the German Primate Center (Göttingen, Germany). Animals were housed individually in air-conditioned facilities on a 12 hr/12 hr light/dark cycle with artificial illumination (lights on from 8:00 A.M. to 8:00 P.M.) and with free access to food and water (Fuchs E. “Tree shrews”, In: Poole T, (ed) UFAW handbook on the care and management of laboratory animals Oxford: Blackwell, 235-245, 1999). All treatments were performed during the day (activity period, lights on). Animal experiments were conducted in accordance with the European Communities Council Directive of Nov. 24, 1986 (86/EEC) and were approved by the Government of Lower Saxony, Germany.

Animals received the NK₁ receptor antagonist Compound 1 orally. A pilot study to establish the dose of Compound 1 that blocks NK₁ receptors in the tree shrew brain was conducted. The methodology used is based on the ability of NK₁ receptor antagonists to block nicotine-induced vomiting in musk shrews (Tattersall et al., Neuropharmacology, 34, 1697-1699, 1995). Adult tree shrews (n=4) received either vehicle or Compound 1 in different dosage orally followed 30 minutes later by subcutaneous administration of (−)-nicotine (4 mg/kg; Sigma), and the number of emetic episodes occurring during the following 30 min was recorded. Each animal received each treatment in a crossover design, with 10 days washout period between studies. Emetic episodes were abolished by treatment with 20 mg/kg per day Compound 1, whereas lower doses, i.e. 5 or 10 mg/kg per day and vehicle were ineffective. These results showed that treatment with 20 mg/kg per day Compound 1 could effectively block central NK₁ receptors.

The experimental design was as follows: Animals were divided into three experimental groups: ‘Control’, ‘Stress’ and ‘Stress+Compound 1’, each consisting of six tree shrews. The experiment consisted of three different phases and lasted for six weeks (42 days). The first experimental phase went on for 7 days, during which all animals remained undisturbed and body weight was recorded daily. The second phase of the experiment was a 7-day period, during which the animals of the Stress' and the ‘Stress+Compound 1’ group were submitted to daily psychosocial conflict. For the induction of psychosocial conflict one naive male was introduced into the cage of a socially experienced male. After establishment of a clear dominant/subordinate relationship, the two animals were separated by a wire mesh barrier. As in earlier studies (Fuchs et al., Pharmacol. Biochem. Behav. 54, 219-228, 1996; Kramer et al., Neurosci. Biobehav. Rev. 23, 937-947, 1999 and Czeh et al., Proc. Natl. Acad. Sci. USA 98, 12796-12801, 2001), all of the naive animals became subordinate. The barrier was removed every day for approximately 1 hour allowing physical contact between the two males only during this time. By this procedure, the subordinate animal was protected from repeated attacks, but it was constantly exposed to olfactory, visual and acoustic cues from the dominant. Under these conditions, subordinate animals displayed characteristic subordination behavior such as reduced locomotor activity and marking behavior. The third experimental phase consisted of the antidepressive drug treatments lasting for 28 days. During this time the stressed animals remained in the psychosocial conflict situation and were treated daily with the antidepressant drugs or vehicle, respectively. The animals of the ‘Stress+Compound 1’ group received the compound (20 mg/kg/day) orally in the morning between 8:00 and 8:15 A.M. In all cases, the drug solutions were freshly prepared every second day and stored light protected and cool. Animals of the Stress group were treated according to the same experimental schedule but received tap water only. The animals of the Control group were individually housed and undisturbed in separate quarters elsewhere in the animal facility and received normal tap water.

Determination of Testosterone Concentrations

For determination of testosterone, 100 μl serum was two times extracted with 1 ml of diethylether by vortexing for 10 min. Following extraction, the combined ether phases were evaporated under a stream of N₂ and dried extracts reconstituted in 300 μl assay buffer (PBS, containing 0.1% BSA, pH 7.0). 50 μl aliquots of reconstituted extracts were then measured for concentrations of testosterone by enzyme-immunoassay as described by Kraus et al. (Physiol. Behav. 66: 855-861, 1999).

Dose

From the binding affinity measured for a given compound, one can estimate a theoretical lowest effective dose. At a concentration of the compound equal to twice the measured Kᵢ-value, nearly 100% of the neurokinin receptors likely will be occupied by the compound. Converting that concentration to mg of compound per kg of patient yields a theoretical lowest effective dose, assuming ideal bioavailability. Pharmacokinetic, pharmacodynamic, and other considerations may alter the dose actually administered to a higher or lower value. The dosage expediently administered is 0.001-1000 mg/kg, preferably 0.1-100 mg/kg of patient's bodyweight.

Treatment

The term “treatment” as used herein refers to any treatment of a human condition or disease and includes: (1) preventing the disease or condition from occurring in a subject which may be predisposed to the disease but has not yet been diagnosed as having it, (2) inhibiting the disease or condition, i.e., arresting its development, (3) relieving the disease or condition, i.e., causing regression of the condition, or (4) relieving the conditions caused by the disease, i.e., stopping the symptoms of the disease.

Testosterone Levels

The concentration of testosterone in plasma of adult men ranges from 300 to 1,200 ng per 100 ml (mean 650 ng per 100 ml), and the concentration in women varies from 34 to 60 ng per 100 ml (mean 54 ng per 100 ml) (M. B. Lipsett in: “Cecil Textbook of Medicine” 15^{th} edition, page 2165, P. B. Beeson, W McDermott and J. B. Wyngaarden (editors)). Within the context of this application a “deficient testosterone level” is a level below the normal lower limit of the testosterone plasma level, thus below 34 ng per 100 ml for women, and below 300 ng per 100 ml for men.

Conditions Associated with Low Testosterone Levels

For instance in women, hypertension and obesity have been associated with testosterone deficiency (see above). There are however, many causes of both hypertension and obesity.

The present invention is based on the discovery that neurokinin-1 receptor antagonists induce a profound increase in serum testosterone levels in animals. For that reason they will become valuable in the treatment of conditions associated with low testosterone levels, in those patients having deficient testosterone levels.

Thus, the present invention for instance relates to the use of neurokinin-1 receptor antagonists in a sub-population of patients diagnosed with hypertension and which have deficient testosterone levels.

### EXAMPLE

### Pharmacological Test Results

Effects of chronic psychosocial stress and concomitant Compound 1 treatment on serum testosterone concentrations.

| group | Serum testosterone concentration (ng/ml) |
|---|---|
| Control | 2.67 ± 0.55 (n = 6) |
| Stress | 1.08 ± 0.24 * (n = 6) |
| Stress + Compound 1 | 6.45 ± 2.00 *# (n = 6) |

Serum testosterone levels were measured from blood samples collected before perfusing the animals. Serum testosterone concentrations were significantly (p<0.05) decreased in animals submitted to chronic stress, whereas treatment with the NK₁ receptor antagonist Compound 1 induced a profound increase of testosterone levels: animals of the ‘Stress+Compound 1’ group had a six times higher (p<0.05) testosterone levels compared to the ‘Stress’ group, and even if compared to ‘Control’ animals, Compound 1 treatment resulted in a two fold increase (p<0.05) in serum testosterone concentrations. One-way ANOVA followed by Newman-Keuls test as post-hoc analysis: *p<0.05 versus Control, # p<0.05 versus Stress.

1. Use of a neurokinin-1 receptor antagonist for the preparation of a pharmaceutical composition for treating conditions requiring testosterone replacement.

2. Use of a neurokinin-1 receptor antagonist for the preparation of a pharmaceutical composition for treating conditions associated with low testosterone levels, in patients having deficient testosterone levels.

3. Use as claimed in claim 2, characterized in that said deficient levels are levels below the normal lower limit of the testosterone plasma concentration.

4. Use as claimed in claim 3, characterized in that said lower limit is 34 ng per 100 ml for women, and 300 ng per 100 ml for men.

5. Use of a neurokinin-1 receptor antagonist for the preparation of a pharmaceutical composition for treating of hypogonadism in men.

6. Use as claimed in any of the claims 1-5, characterized in that said neurokinin-1 antagonist is a compound with the general formula (I) 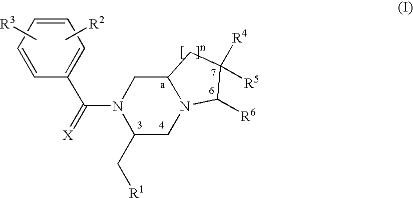
wherein:
R¹ represents phenyl, 2-indolyl, 3-indolyl, 3-indazolyl or benzo[b]thiophen-3-yl, optionally substituted with halogen or alkyl (1-3C),
R² and R³ independently represent halogen, H, OCH₃, CH₃ and CF₃,
R⁴, R⁵ and R⁶ independently represent H, OH, O-alkyl(1-4C), CH₂OH, NH₂, dialkyl(1-3C)N, pyrrolidin-1-yl, piperidin-1-yl, morpholin-4-yl or morpholin-4-yl, substituted with one or two methyl or methoxymethyl groups, morpholin-4-ylamino, morpholin-4-ylmethyl, imidazol-1-yl, thiomorpholin-4-yl, 1,1-dioxo-thiomorpholin-4-yl or 3-oxa-8-azabicyclo[3.2.1]oct-8-yl; R⁴ and R⁵ together may represent a keto, a 1,3-dioxan-2-yl or a 1,3-dioxolan-2-yl group,
x represents either O or S,
n has the value of 1, 2 or 3,
a is the asymmetrical carbon atom 8a, 9a or 10a when n equals 1, 2 or 3 respectively.

7. Use as claimed in any of the claims 1-5, characterized in that said neurokinin-1 antagonist is a compound with the general formula (II) 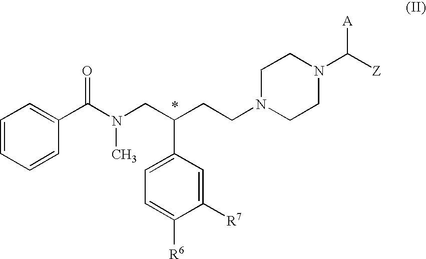
wherein
A is naphthyl, phenyl optionally substituted by hydroxy, mono- or bicyclic heteroaryl or C₃₋₆-alkenyl optionally substituted by phenyl,
Z stands for a subgroup of the general formula 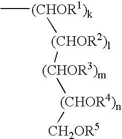
wherein
R¹ is hydrogen or lower alkanoyl, or together with another substituent, selected from the group consisting of R², R³, R⁴ and R⁵, may form a 5- or 6-ring bridged by carbonyl, thiocarbonyl or by methylene optionally substituted by lower alkyl or C₄₋₅-alkylene,
R² is hydrogen or lower alkanoyl, or together with another substituent, selected rom the group consisting of R¹, R³, R⁴ and R⁵, may form a 5- or 6-ring bridged by carbonyl, thiocarbonyl or by methylene optionally substituted by lower alkyl or C₄₋₅-alkylene,
R³ is hydrogen or lower alkanoyl, or together with another substituent, selected from the group consisting of R¹, R², R⁴ and R⁵, may form a 5- or 6-ring bridged by carbonyl, thiocarbonyl or by methylene optionally substituted by lower alkyl or C₄₋₅-alkylene,
R⁴ is hydrogen or lower alkanoyl, or together with another substituent, selected from the group consisting of R¹, R², R³ and R⁵, may form a 5- or 6-ring bridged by carbonyl, thiocarbonyl or by methylene optionally substituted by lower alkyl or C₄₋₅-alkylene,
R⁵ is hydrogen or lower alkanoyl, or together with another substituent, selected from the group consisting of R¹, R², R³ and R⁴, may form a 5- or 6-ring bridged by carbonyl, thiocarbonyl or by methylene optionally substituted by lower alkyl or C₄₋₅-alkylene,
k is 0 or 1,
l is 0 or 1,
m is 0 or 1,
n is 0 or 1,
R⁶ is halogen or hydrogen, and
R⁷ is halogen or hydrogen.

8. Use as claimed in any of the claims 1-5, characterized in that said neurokinin-1 antagonist is a compound with the general formula (III) 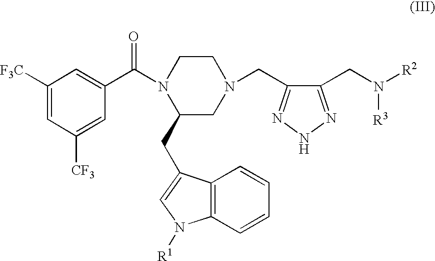
wherein
R¹ is hydrogen or lower alkyl,
R² is lower alkyl, di-lower-alkylamino lower alkyl, lower-alkoxycarbonyl lower alkyl; cyclo(hetero)alkyl having 5-6 ring atoms, which may optionally be substituted once or twice by lower alkyl and which optionally contains 1-2 double bonds; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl or by spiro-C₄-C₅-alkylene; or phenyl lower alkoxy optionally substituted once or twice in the phenyl ring by halogen, lower alkyl and/or lower alkoxy, and
R³ is lower alkyl, lower-alkoxycarbonyl lower alkyl or cyclo(hetero)alkyl with 5-6 ring atoms which is optionally substituted once or twice by lower alkyl, or
R² and R³, together with the nitrogen to which they are bonded, form a cyclic group of formula a, 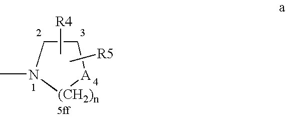
wherein
A is nitrogen, oxygen, methylene or methylidene, the double bond of which, together with the adjacent carbon, is formed in position 3 of group a,
n is a whole number from 1 to 3,
R⁴ is hydrogen, lower alkyl, lower-alkoxy lower alkyl, lower alkoxycarbonyl, lower-alkoxycarbonyl lower alkyl, di-lower-alkylamino lower alkyl; (hetero)phenyl optionally substituted once or twice by halogen, lower alkyl and/or lower alkoxy; (hetero)phenyl lower alkyl optionally substituted once or twice in the (hetero)phenyl ring by halogen, lower alkyl and/or lower alkoxy, the lower-alkyl chain of which (hetero)phenyl lower alkyl is optionally substituted once or twice by lower alkyl; cyclo(hetero)alkyl with 5-6 ring atoms, or cyclo(hetero)alkyl lower alkyl, the cyclo(hetero)alkyl group of which has 5-6 ring atoms, and
R⁵ is hydrogen, lower alkyl or lower-alkoxy lower alkyl, or
R⁴ and R⁵ together are spiroethylenedioxy bonded to a carbon of group a; C₃-C₄-alkylene bonded to two adjacent atoms of group a; or phenyl fused via two adjacent carbons of group a, or
R² and R³, together with the nitrogen to which they are bonded, form a pyrrolidine ring which is substituted twice by C₄-alkylene which is bonded each time via two adjacent carbon atoms,

9. Use as claimed in any of the claims 1-5, characterized in that said neurokinin-1 antagonist is a compound with the general formula (IV) 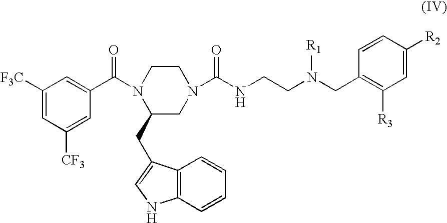
wherein
R₁ is hydrogen or lower alkyl,
R₂ is hydrogen or halogen and
R₃ is hydrogen or lower alkoxy,

10. Use as claimed in any of the claims 1-5, characterized in that said neurokinin-1 antagonist is a compound with the general formula (V) 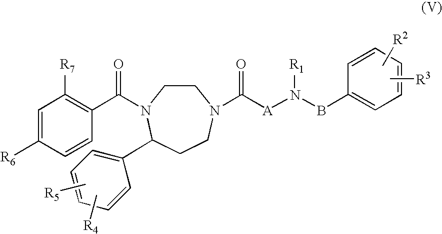
wherein
R¹ is hydrogen or lower alkyl,
R² is hydrogen, lower alkyl, lower alkoxy, halogen or trifluoromethyl, and
R³ is hydrogen, lower alkyl, lower alkoxy, halogen or trifluoromethyl, or
R² and R³ together are alkylenedioxy with 1 to 2 carbon atoms, bonded to adjacent carbon atoms of the phenyl ring,
R⁴ is hydrogen, lower alkyl, lower alkoxy, halogen or trifluoromethyl, and
R⁵ is hydrogen, lower alkyl, lower alkoxy, halogen or trifluoromethyl, or
R⁴ and R⁵ together are alkylenedioxy with 1 to 2 carbon atoms, bonded to adjacent carbon atoms of the phenyl ring,
R⁶ is lower alkyl, halogen or trifluoromethyl,
R⁷ is lower alkyl, halogen or trifluoromethyl,
A is a —(CH₂)ₙ— group in which n stands for an integer from 1 to 3, or an —NH—(CH₂)ₘ— group in which m stands for an integer from 2 to 3, and
B is an alkylene chain with 1 to 3 carbon atoms, optionally substituted by lower alkyl.

11. Use as claimed in any of the claims 1-5, characterized in that said neurokinin-1 antagonist is a compound with the general formula (VI) 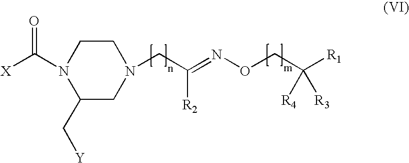
wherein:
x represents phenyl or pyridyl substituted with 1 or 2 substituents from the group CH₃, CF₃, OCH₃, halogen, cyano and 5-CF₃-tetrazol-1-yl
Y represents 2- or 3-indolyl, phenyl, 7-aza-indol-3-yl or 3-indazolyl, 2-naphthyl, 3-benzo[b]thiophenyl or 2-benzofuranyl, which groups may be substituted with one or more halogen or alkyl (1-3C)
n has the value 0-3
m has the value 0-2
R₁ represents NH₂, NH-alkyl (1-3C), dialkyl (1-3C)N, morpholino or morpholino substituted with one or two CH₃ and/or methoxymethyl groups, thiomorpholino 1,1-dioxothiomorpholino, 2-, 3- or 4-pyridyl or 4-CH₃-piperazinyl
R₂ is hydrogen, alkyl (1-4C) or phenyl, or R₂ together with (CH₂)ₘ wherein m is 1, and the intermediate carbon, nitrogen and oxygen atoms forms an isoxazolyl or a 4,5-dihydroisoxazolyl group,
R₃ and R₄ independently represent hydrogen or methyl, or
R₃ and R₄ together are oxygen.

12. Use as claimed in any of the claims 1-5, characterized in that said neurokinin-1 antagonist is a compound with the general formula (VII) 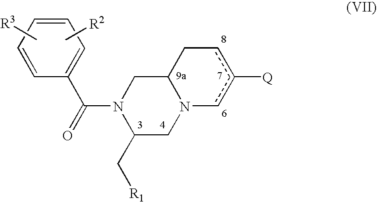
wherein:
R¹ represents phenyl, 2-indolyl, 3-indolyl, 3-indazolyl or benzo[b]thiophen-3-yl, which groups may be substituted with halogen or alkyl (1-3C),
R² and R³ independently represent halogen, H, OCH₃, CH₃ and CF₃,
Q represents an optionally substituted aromatic or heteroaromatic five- or six-membered ring,
the pyrido[1,2-a]pyrazine moiety may or may not contain a double bond between either carbon atoms 6 and 7 or between carbon atoms 7 and 8.

13. Use as claimed in any of the claims 1-5, characterized in that said neurokinin-1 antagonist is a compound with the formula:

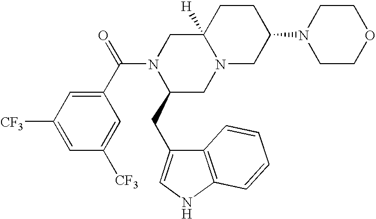

14. Use as claimed in any of the claims 1-5, characterized in that said neurokinin-1 antagonist is a compound with the formula:

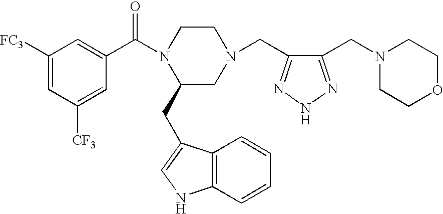

15. Use as claimed in any of the claims 1-5, characterized in that said neurokinin-1 antagonist is a compound with the formula:

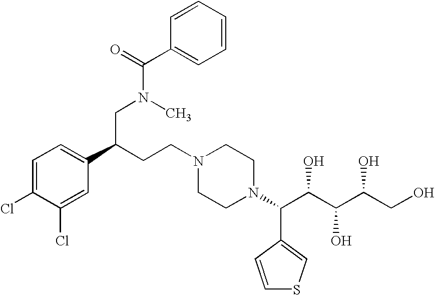

16. Use as claimed in any of the claims 1-5, characterized in that said neurokinin-1 antagonist is a compound with the formula:

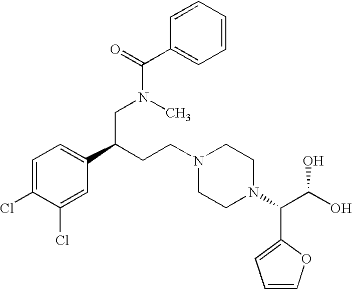

17. Use as claimed in any of the claims 1-5, characterized in that said neurokinin-1 antagonist is a compound with the formula:

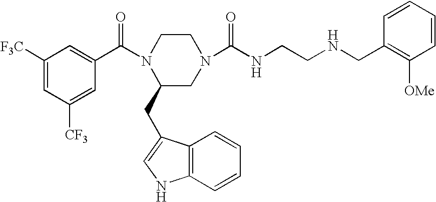

18. Use as claimed in any of the claims 1-5, characterized in that said neurokinin-1 antagonist is a compound with the formula:

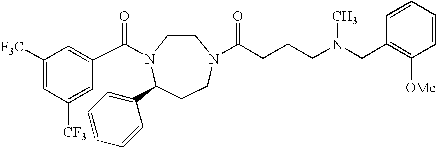

19. Use as claimed in any of the claims 1-5, characterized in that said neurokinin-1 receptor antagonist is selected from anthrotainin, aprepitant, AVE-5883, BIIF-1149, BL-1832, BL-1833, CAM-2445, CAM-6108, capsazepine, CGP-47899, CGP-49823, CGP-73400, cizolirtine, CJ-17493, CP-0364, CP-0578, CP-100263, CP-122721, CP-96345, CP-98984, CP-99994, dapitant, DNK-333, E-6006, ezlopitant, FK-224, FK-355, FK-888, FR-113680, GR-138676, GR-203040, GR-71251, GR-82334, GW-597599, GW-679769, GW-823296, isbufylline, KRP-103, L-161644, L-161664, L-709210, L-732138, L-733060, L-736281, L-737488, L-740141, L-741671, L-742311, L-742694, L-743986, L-756867, L-758298, lanepitant, LY-297911, LY-306740, MDL-105172A, MEN-10930, MEN-11149, MEN-11467, NIP-530, NKP-608, nolpitantium besilate, PSI-697, R-1124, R-116301, R-673, RP-67580, RP-73467, RPR-107880, RPR-111905, S-116474, S-18523, S-19752, Sch-60059, SDZ-NKT-343, SP-PE toxin, SSR-240600, TAK-637, TKA-457, vofopitant, WIN-51708, WIN-64745, WIN-64821, WIN-66306, WIN-67689, WIN-68577, WS-9326A, YM-44778, YM-49244, ZD-4794 and ZD-6021
